(19) United States
(12) Patent Application Publication
(10) Pub. No.: US 2018/0036137 A1
(43) Pub. Date: Feb. 8, 2018

(54) INTERVERTEBRAL CAGES AND THEIR USES

(71) Applicant: SPINEART SA, Meyrin (CH)

(72) Inventor: Jerome LEVIEUX, Satigny (CH)

(21) Appl. No.: 15/554,665

(22) PCT Filed: Mar. 4, 2016
(86) PCT No.: PCT/EP2016/054625

§ 371 Date: Aug. 30, 2017

(30) Foreign Application Priority Data
Mar. 6, 2015 (FR) 15 51917

(51) Int. Cl. A61F 2/44; A61B 17/70

(52) U.S. Cl. CPC A61F 2/447; A61B 17/70; A61F 2/4405; A61F 2002/30538

(57) ABSTRACT

A cage includes an inner male component, which is annular, and an outer female component, which is also annular, the upper surface of the inner component and the lower surface of the outer component forming outer surfaces that define the height of the cage, the inner component engaging in the outer component and being able to slide in relation to the outer component and having a blocking device for blocking a longitudinal displacement of the inner component in relation to the outer component, the cage being provided with a device which adjusts the height and acts individually on one end and the other end of the longitudinal axis of the inner component, a short side of the inner component and a corresponding short side of the outer component each being provided with a slot for the insertion of an adjustment tool.

The present invention relates to intervertebral cages, also called intersomatic cages, and to their uses.

In humans, degeneration of the intervertebral disc tends to reduce the space within the disc and to narrow the foramina through which the nerve roots issue from the spinal canal. The intervertebral discs, it will be remembered, are located in the vertebral column between two consecutive vertebrae. An intervertebral disc comprises a ring of cartilage provided at its center with a gelatinous nucleus. The intervertebral discs are elastic and thus help absorb the shocks to which a vertebral column is subjected.

The surgical treatment of this type of disease of the spine may require one or more vertebral segments to be fused in the best possible anatomical position. The technique requires polishing the vertebral plates and employing a bone graft in order to promote fusion of the vertebrae. In order to restore the normal space and the anatomical sagittal angle of the segment (lordosis), intersomatic cages are implanted between the vertebrae, bone graft can be placed in the cage, and sometimes round about it, in order to bring about the fusion. Intersomatic cages are generally designed as a casing with two slots, one on the lower face and one on the rear face, the lateral faces also being able to be openworked. These cages are sometimes not hollow but solid, in which case no bone graft will be placed in the inside. In the text below, the “height” of a cage is understood as the dimension measured in the direction corresponding to the thickness (or height) of an intervertebral disc.

Expandable cages and “lordosis adjustment” cages have recently appeared on the market. Examples of expandable cages are the Latis® or Caliber® cages from the company Globus. Examples of the lordosis adjustment cages are the Varlock® cages from the company Kisco.

The disadvantages of these cages are of several kinds:

The technology used to permit their function is expensive since it is complex and often involves several small components, and this leads to implants that are costly to manufacture and thus limits their accessibility to the market.

None of these cages is a lordosis adjustment cage and expandable cage at one and the same time. However, the surgeon wishes to restore both the normal space and the anatomical sagittal angle of the segment (lordosis).

Their complex technology, resulting from the presence of screws with very small diameters and of sliding components, increases the risks of malfunction and thus increases the risks for the patient.

Their complex technology does not allow enough space to be formed inside the cage in order to insert a sufficient quantity of bone graft.

It would therefore be desirable to have intervertebral cages that are
very simple, hence inexpensive to produce, and more reliable than complex cages,
capable of providing both the lordosis adjustment and the lift,
provided with a considerable volume available to receive bone graft.

The applicant has designed intervertebral cages that entirely satisfy the aforementioned criteria.

It is for this reason that the present application relates to an intervertebral cage comprising two components, namely
an annular inner male component, which has two short sides and two long sides, hence four outer faces and four inner faces, and an upper surface and a lower surface, and
an outer female component, which is likewise annular and has two short sides and two long sides, hence four outer faces and four inner faces, and an upper surface and a lower surface,
   the upper surface of the annular inner male component and the lower surface of the annular outer female component forming outer surfaces that determine the height of the cage,
   said annular inner male component engaging and preferably wedging in the annular outer female component, each of these two components having the general shape of a parallelepiped, hence a longitudinal axis, the annular inner male component being able to slide with respect to the annular outer female component and having a device for blocking a longitudinal displacement of the annular inner male component with respect to the annular outer female component,
   the intervertebral cage being provided with a device which adjusts the height of the cage and acts individually on one end and the other end of the longitudinal axis of the annular inner male component,
   a short side of the annular inner male component and a corresponding short side of the annular outer female component each being provided with a slot for the insertion of an adjustment tool which passes through the annular outer female component in order to actuate the annular inner male component,
   as a consequence of which it is possible to increase the height of the cage and modify the angle between the outer surfaces.

Under preferred conditions of implementation of the invention, one or more sides, preferably one side, of the annular outer female component is provided with a slot in order to permit displacement of the annular inner male component, so as to increase the height of the cage and modify the angle between the outer surfaces. The slot is preferably provided in a short side of the annular outer female component. Advantageously, the annular inner male component is likewise provided on one or more sides, preferably on only one side, with a slot that can be used likewise for this purpose.

A suitable tool can advantageously be used for this purpose. A suitable tool is, for example, a screwdriver or a wrench acting directly or indirectly on the annular inner male component, for example (indirect action) on a device provided for adjusting the height of the cage and comprising two elongate rotary shafts with concentric axes, each shaft having a cam with flattened segments and being able to be turned independently of the other. By turning both, it is possible to increase the height of the cage in an equal manner along the longitudinal axis. By turning only one of the two, the height is increased on only the side of the longitudinal axis where the cam acts, and it is thus possible to obtain lordosis. By turning one more than the other, it is possible to simultaneously increase the height of the cage and produce lordosis.

The inner male component and the outer female component are annular and therefore open. The opening of the outer female component permits the insertion of the inner male component. Each of these two components has the general shape of a parallelepipedal annulus.

In horizontal section, if the cages are considered when arranged in the intervertebral space of an individual who is standing up, the general shape of the cages is rectangular. They also have an upper surface and a lower surface. The upper surface of one and the lower surface of the other form external surfaces that determine the height of the cage.

In the cages according to the invention having the general shape of a parallelepiped, it is thus possible to distinguish, as in any rectangle, two short sides and two long sides. The annular nature of the components allows four outer faces and four inner faces to be distinguished.

As has been seen above, the annular inner male component is intended to engage in the annular outer female component.

The annular inner male component is able to slide with respect to the annular outer female component and has a device for blocking the longitudinal displacement of the annular inner male component with respect to the annular outer female component. For this purpose, for example, the outer face of the short sides of the inner male component is convex and bears on the inner face of the short sides of the annular outer female component. As the short sides of the inner male component are convex, the inner male component can be displaced according to pitching movements. This convex shape can result from a dome shape, for example a hemispherical shape, of said short sides. It can also result from a semi-cylindrical surface, that is to say a semi-circular gutter shape (like a traditional gutter of a house). In the latter case, the gutter is arranged horizontally.

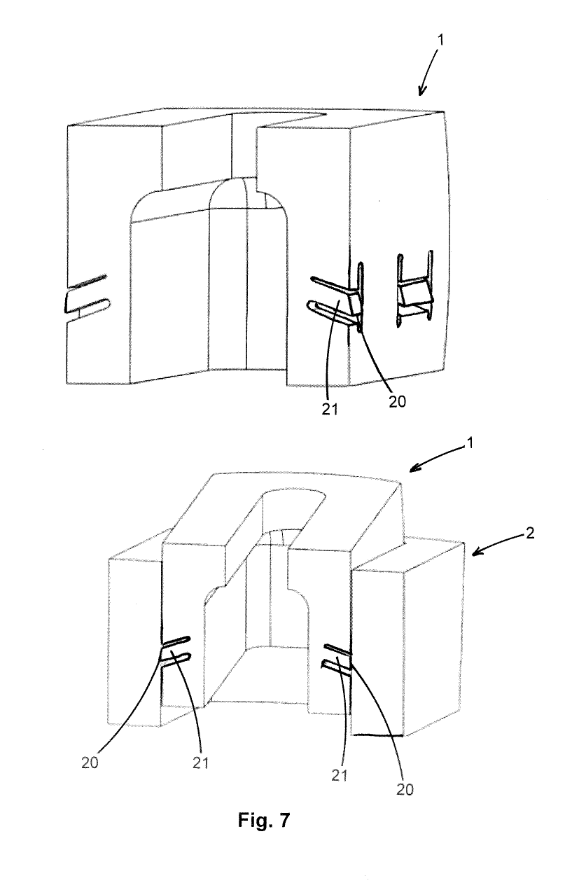

When the short sides of the annular inner male component have the shape of a semi-cylindrical surface, the inner surfaces of the short sides of the annular outer female component 2 are plane faces, as shown in FIG. 6.

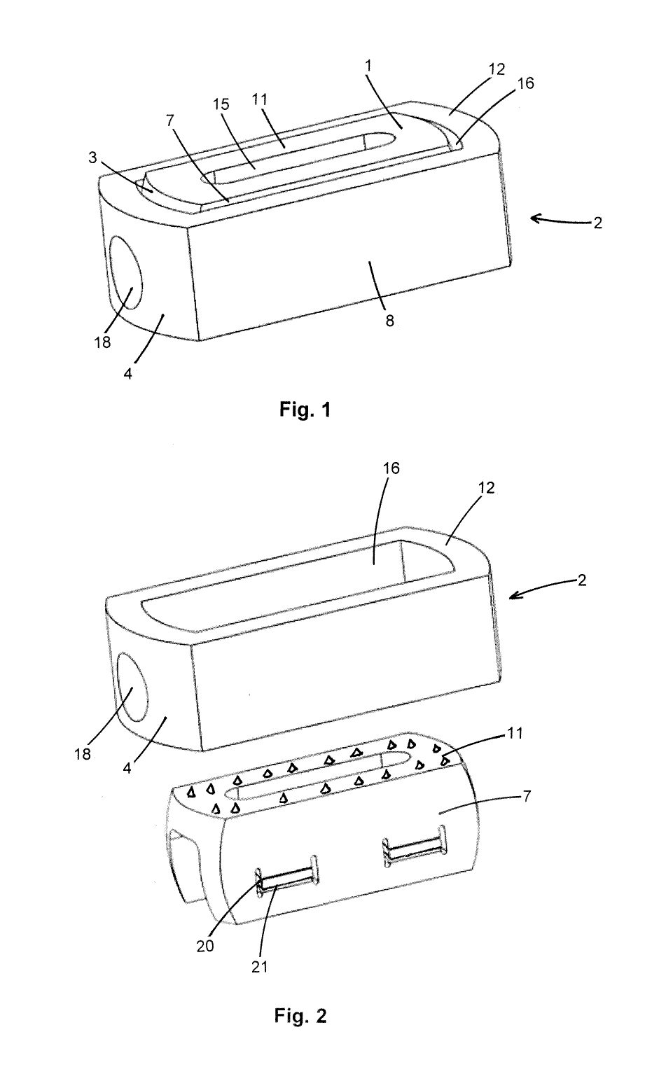

When the short sides of the annular inner male component have the dome shape, in this case the inner surface of the short sides of the outer female component has the shape of semi-circular gutters that are arranged vertically, as shown in FIGS. 1 to 5.

In summary, it may be said that the outer face of the short sides of the inner male component bears on the inner face of the short sides of the annular outer female component and that the short sides of the inner male component are capable of describing a circle of radius (R) while remaining in contact with the inner surface of the short sides of the outer female component. This configuration allows the assembly to withstand the shearing forces when the cage is opened and there is lordosis. Expressed very simply, it may be said that arcs arranged on each side of the cage describe substantially the same theoretical circle.

With a view to blocking the longitudinal displacement of the annular inner male component with respect to the annular outer female component, it is also possible for pins to be arranged, preferably in a median position, in the long sides of the inner male component, these pins being able to cooperate with an oblong hole passing through each of the long sides of the outer female component.

According to the present invention, the intervertebral cage is provided with a device which adjusts the height of the cage and acts individually on one end and the other end of the longitudinal axis of the annular inner male component. This device can take various forms.

In one embodiment of a device for adjusting the height of the cage by acting individually on one end and the other end of the longitudinal axis of the annular inner male component, where the adjustment is reversible, a system according to the invention comprises a device comprising two elongate rotary shafts with concentric and independent axes, each shaft having a cam with flattened segments and being able to be turned independently of the other. The flattened segments of the cam are spaced progressively farther apart from the axis. The blocking is achieved each time a flattened segment comes to bear against the annular inner male component. The flat faces serve both to adjust the height and also to keep the cage in the desired position via the choice of a front and rear flat face.

By turning both shafts, it is possible to increase the height of the cage in an equal manner along the longitudinal axis. By turning only one of the two shafts, the height is increased on only the side of the longitudinal axis where the cam acts, and it is thus possible to obtain lordosis. By turning one more than the other, it is possible to simultaneously increase the height of the cage and produce lordosis.

In an embodiment in which the adjustment is irreversible, the outer faces of the long sides of the annular inner male component or the inner faces of the long sides of the annular outer female component, or all of these faces, are each provided with two or more devices that each constitute a system of elastic engagement. A system of irreversible elastic engagement is a type of assembly in which one or both elements of the assembly is/are deformed during introduction. After introduction, the elements can no longer be separated. According to the invention, this system is provided with multiple positions (or notches). Thus, several adjustments are possible and, for each adjustment, a return is no longer possible.

The presence of these systems allows the height of the cage and the angle between the outer surfaces to be varied irreversibly.

For example, two devices, spaced apart from each other, are located on each long side. For example, each device is preferably arranged near a short side.

The system of irreversible engagement also comprises fixation by wedging. In this embodiment, for example, the outer faces of the long sides of the annular inner male component are provided with a pair of flexible tabs, each of these being provided with a blade protruding from the outer general surface of said long sides. Such a configuration allows the two components to be maintained relative to each other by wedging, without the need to provide notches in the inner surface of the long sides of the annular outer female component, as shown in FIG. 7 for example.

Such a device, according to another embodiment, comprises for example a series of notches that are formed by several inclined blades arranged parallel to each other. The system is made irreversible by using a surface perpendicular to the direction of movement of disassembly, while keeping a contact surface inclined in the direction of introduction.

Such devices are preferably arranged to permit height adjustment in steps of 0.3 to 3 millimeters, preferably 0.5 to 2.5 millimeters, particularly 1 to 2 millimeters. Fine height adjustment and sufficient strength are thus both obtained.

Two or more devices that each constitute a system of elastic engagement are located opposite each other on each long side. For example, there are four devices per side, but preferably three devices, or particularly two devices per side, as shown in FIG. 2.

In a system according to the invention, the devices are arranged opposite each other in pairs so as to form the system of irreversible engagement.

In such a system of engagement, at least one device, preferably just one of the two devices arranged opposite each other, is elastic. For this purpose, this device is advantageously mounted on an elastic tab. It is also possible to have a tooth on one side and a series of notches on the opposite side, or two series of notches opposite each other.

Depending on the height of the one or more notches in which the one or more opposite notches are arranged, it is possible to irreversibly vary the height of the cage and the angle between the outer surfaces.

It will be noted that the first embodiment of the height adjustment of the cage, using 2 series of flat faces, is compatible with the other adjustment systems mentioned above and can therefore be used jointly.

An expandable cage according to the invention can be made of any biocompatible material appropriate to this type of implant. For example, it can be made of titanium, titanium alloy, PEEK, stainless steel, cobalt-chromium alloy, tantalum, etc.

Furthermore, the upper and lower surfaces, intended to be in contact with the bone of the adjacent vertebral plates, will be able to be covered with materials that facilitate bone attachment, for example porous titanium or hydroxyapatite. They will also be able to be produced using technologies for obtaining what is called a “trabecular” surface, that is to say this surface is porous and has characteristics close to those of human bone (pore diameters and porosity).

The thickness (or height) of a cage according to the invention, with reference to the end notches, can measure 4 to 16 mm, preferably 5 to 14 mm, particularly 6 to 12 mm.

The expansion of a cage according to the invention can be 0 to 8 mm, preferably 0 to 4 mm, preferably 0 to 2 mm.

In the antero-posterior direction of the human body, the dimension of the cage can measure 10 mm to 50 mm, preferably 12 to 45 mm, particularly 14 to 40 mm.

In the transverse direction (from left to right) of the human body, the dimension of the cage can measure 5 mm to 25 mm, preferably 7 to 20 mm, particularly 8 to 12 mm.

The surfaces in contact with the vertebrae can have a skewed shape, for example a dome shape or tile shape, and are preferably flat.

By virtue of the design according to the present invention, the outer surfaces can be angled with respect to each other. The angle between the contact surfaces, which is conferred by the use of the end adjustments, can be 0 to 25 degrees, preferably 0 to 20 degrees, particularly 0 to 12 degrees.

It will be remembered that the contact surfaces of the intervertebral cages are usually provided with a roughened finish in order to permit attachment of the implant. Moreover, these surfaces intended to be in contact with the bone of the adjacent vertebral plates will also be able to be covered with materials that facilitate bone attachment, for example porous titanium or hydroxyapatite. They will also be able to be produced using technologies for obtaining what is called a “trabecular” surface, that is to say this surface is porous and has characteristics close to those of human bone (pore diameters and porosity).

The intervertebral cages forming the subject matter of the present invention have very advantageous properties. In particular, like traditional intervertebral cages, they are capable of restoring the normal space between the vertebrae and of using a bone graft to promote fusion of the vertebrae, but also capable of
restoring the normal space by adjusting the height of the cage easily and precisely, and
restoring the anatomical sagittal angle of the segment (lordosis).

They are of a simple structure since, in certain embodiments, they can be produced as 2 components only, and, while being expandable and making it possible to adjust the lordosis, they additionally provide a substantial volume in which to place a sufficient quantity of bone graft for the purpose of fusion.

These properties are illustrated below in the description of the figures. They justify the use of the above-described cages in the replacement of an intervertebral disc that has suffered degeneration as a result of trauma, disease or aging. They also justify the use of the above-described intervertebral cages in a method of fusion between two vertebrae. They likewise justify the use of the above-described intervertebral cages in a method for restoring the anatomical sagittal angle of an intervertebral segment. They equally justify the use of the above-described intervertebral cages in a method for restoring the intervertebral space.

It is for this reason that the present application also relates to a method for replacing an intervertebral disc which, for example, has suffered degeneration as a result of trauma, disease or aging, in which method the two components of an above prosthesis in the non-expanded position are implanted between two adjacent vertebrae, and then a suitable adjustment tool is inserted into the cage by way of the slots and is used to adjust the chosen height or the chosen lordosis or both. The present application likewise relates to a method for fusion between two adjacent vertebrae, in which method at least one above cage is implanted between two adjacent vertebrae (a cervical or lumbar cage by an anterior route and two lumbar cages by a posterior route), and a bone graft is arranged in a free volume of the cage accessible from the outside.

The above methods comprise the steps of
removing an intervertebral disc from between two adjacent vertebrae, for example by an approach through the back or through the abdomen,
filling the empty space created by the removal of the intervertebral disc, by arranging an intervertebral cage according to the invention in said space,
carrying out the necessary adjustment or adjustments to the height or to the lordosis, or to both of these parameters, with the aid of a suitable ancillary instrument,
optionally inserting a bone graft.

The suitable ancillary instrument can, for example, be a simple cylindrical rod, preferably comprising a handle, or a more complex device as described above, which can be actuated by tools such as a flat-head screwdriver, cross-head screwdriver or Allen key.

The preferred conditions of use of the above-described intervertebral cages apply likewise to the other subjects of the invention that are mentioned above, for example to the methods for replacing an intervertebral disc.

The invention will be better understood by reference to the attached drawings, in which:

FIG. 1 shows a schematic perspective view of a cage according to the invention seen from above, in which the inner component is inserted into the outer component,

FIG. 2 shows a view comparable to that of FIG. 1, in which the inner component has not yet been inserted into the outer component, and in which the components have been rotated through 180°,

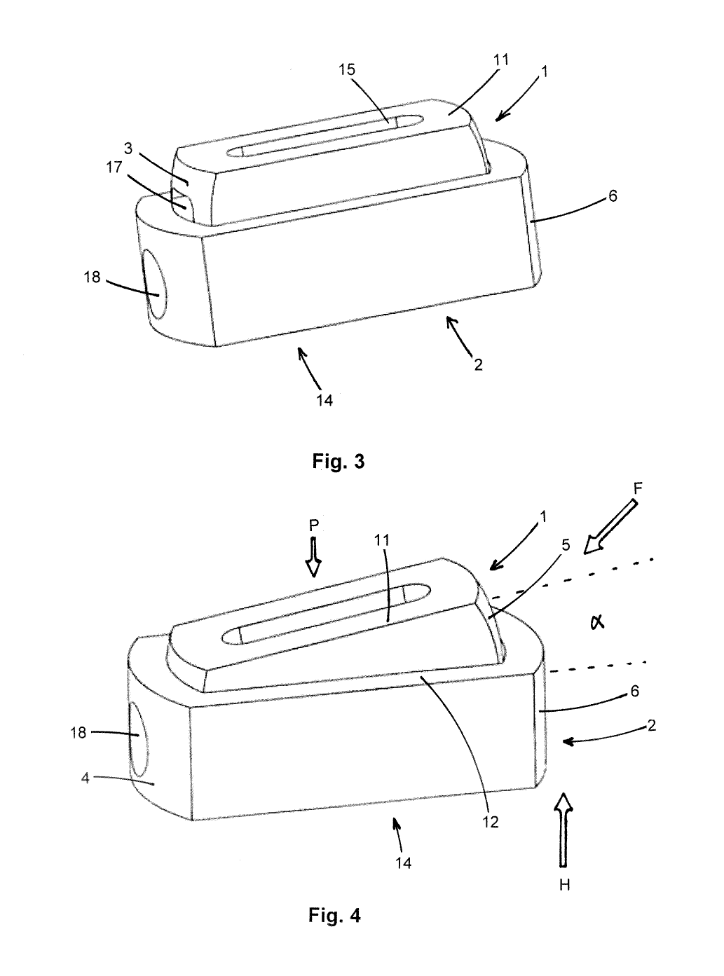

FIG. 3 shows a view comparable to that of FIG. 1, in which the inner component has been raised in order to increase the height of the assembly,

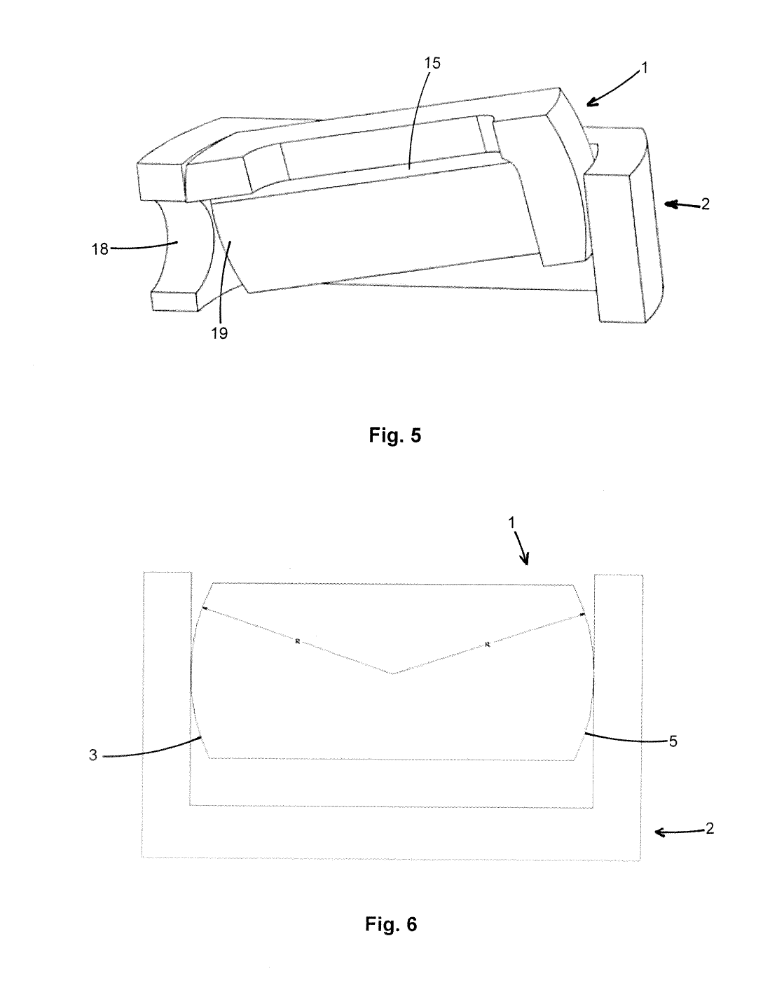

FIG. 4 shows a view comparable to that of FIG. 3, in which the height of the inner component has been increased differentially in order to obtain lordosis,

FIG. 5 shows a longitudinal sectional view of the profile of an intervertebral cage shown in FIG. 4,

FIG. 6 shows a longitudinal sectional profile of an intervertebral cage according to the invention, of the type in which the short sides of the annular inner male component have the shape of a semi-cylindrical surface, while the inner surfaces of the short sides of the annular outer female component 2 are plane faces,

FIG. 7 shows, at the top, a cross-sectional view of an annular inner male component and, at the bottom, a cross-sectional view in which the annular inner male component is arranged in an annular outer female component,

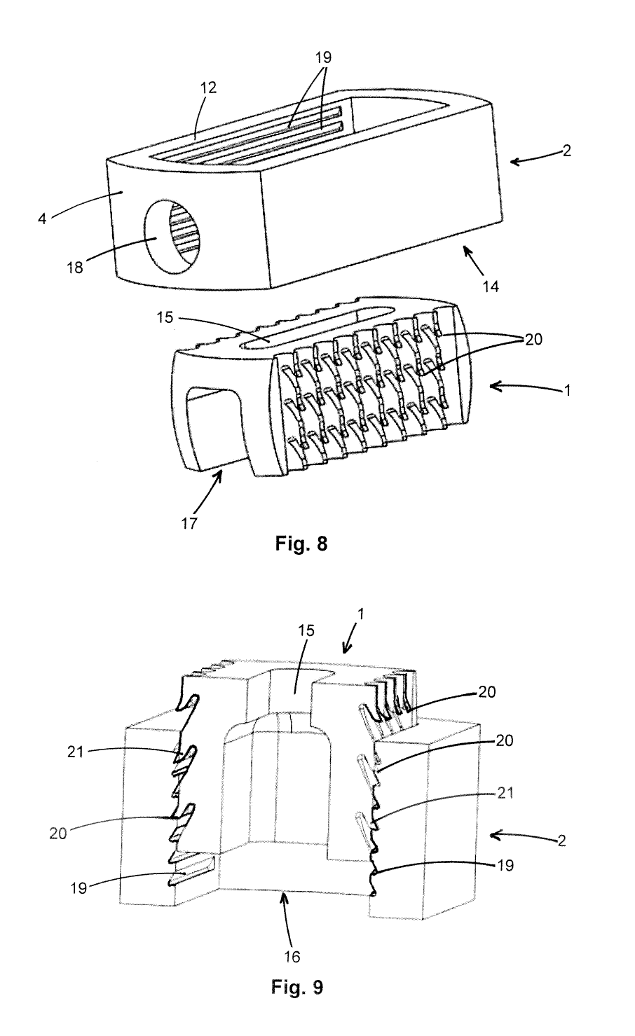

FIG. 8 shows, at the top, a perspective view of an annular outer female component and, at the bottom, a perspective view of an annular inner male component,

FIG. 9 shows a cross-sectional view in which the annular inner male component from FIG. 8 is arranged in the annular outer female component from FIG. 8,

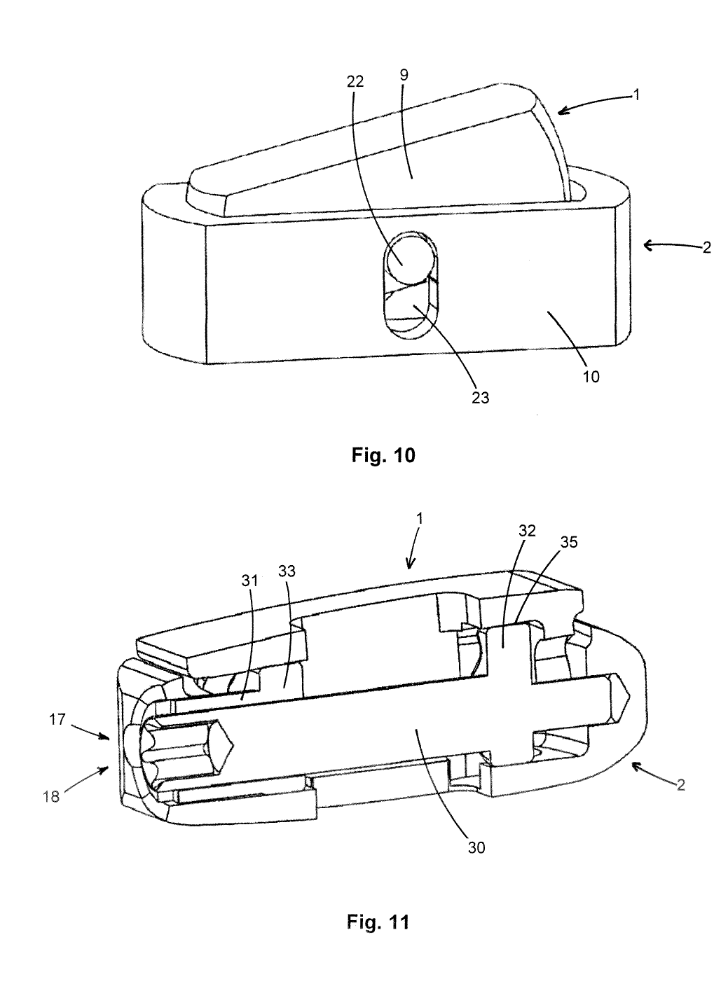

FIG. 10 shows a view similar to that of FIG. 4,

FIG. 11 shows an exploded view of a cage according to the invention in which an adjusting device is installed,

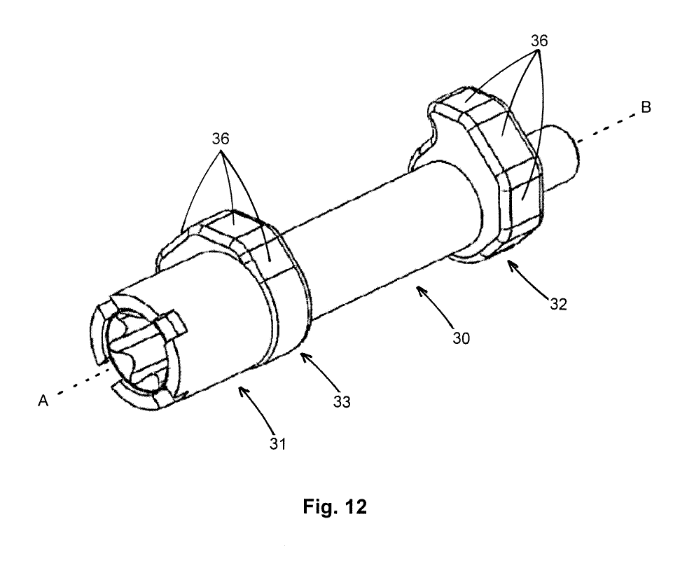

FIG. 12 shows a perspective view of the adjusting device from FIG. 11.

In FIG. 1, the cage is shown in the position in which it is arranged in the intervertebral space of an individual standing up.

It will be noted that a vertebral cage according to the invention is composed of an annular inner male component 1 that is engaged in an annular outer female component 2. The inner male component 1 and the outer female component 2 are annular and thus open at their center. Each of these two components has the general shape of a parallelepipedal annulus and has two short sides 3, 4, 5, 6 and two long sides 7, 8, 9, 10, hence four outer faces and four inner faces, and an upper surface 11, 12 and a lower surface 13, 14, the latter being concealed in this figure. The general shape of these components can also be described as short flattened cylinders. There is thus an opening 15, 16 at the center of these components.

The dimensions and the shape of the opening 16 of the outer female component 2 have permitted the insertion of the inner male component 1.

On account of their general parallelepipedal shape, in horizontal section, the cages have the general shape of a rectangle. They also have an upper surface and a lower surface. The upper surface of one 7 and the lower surface 12 of the other, not visible in this figure, form outer surfaces that determine the height of the cage.

The inner surface of the short sides 4, 6 of the outer female component is convex and forms a vertical gutter. The outer surface of the short sides 3, 5 of the inner male component is convex in a dome shape.

The outer face of the short sides 3, 5 of the inner male component 1 bears on the inner face of the short sides 4, 6 of the annular outer female component 2.

One of the short sides 3 of the annular inner male component 1 is provided with a slot 17 that merges into its opening 15. This slot 17 is not visible in this figure. In the same way, one of the short sides 4 of the annular outer female component 2 is provided with a slot 18 that merges into its opening 16. When a cage according to the invention is used, the two slots are situated on the same side of the cage.

FIG. 2 shows a perspective view of a vertebral cage from FIG. 1, in which the two components are shown separated before the annular inner male component 1 is introduced into and engaged in the annular outer female component 2.

From this view it is possible to discern other particular features of the cage. Each of the outer faces of the long sides of the annular inner male component is provided with a pair of flexible tabs longitudinally spaced apart from each other, each provided with a blade that protrudes from the outer general surface of said long sides. Such a configuration allows the two components to be held relative to each other by sintering, without the need to provide notches in the inner surface of the long sides of the annular outer female component 2.

The upper surface 11 of the annular inner male component 1 is provided with fastening reliefs. The same goes for the lower surface 14 (not visible in this figure) of the annular outer female component 2.

FIG. 3 shows a perspective view of a vertebral cage from FIG. 1, in which the annular inner male component 1 has been introduced farther into the annular outer female component 2. For this reason, it is possible to distinguish the slot 17 formed in a short side 3 of the annular inner male component 1.

FIG. 4 shows a cage according to the invention which, by virtue of the series of blades, has been imparted a specific lordosis (see angle α between the upper surface 11 of the annular inner male component 1 and the surfaces 12, 14 of the annular outer female component 2).

FIG. 5 shows an exploded longitudinal sectional view along the length of a cage similar to the one shown in FIG. 4. It shows better the configuration of the outer surfaces of the short sides of the male component and inner surfaces of the short sides of the annular outer female component 2. The inner surfaces of the short sides of the annular outer female component 2 have the shape of perpendicularly arranged gutters. For their part, the outer surfaces of the short sides of the annular inner male component 1 have a dome shape. While being wedged in horizontal translation between the inner surfaces of the short sides of the annular outer female component 2, the annular inner male component 1 can nevertheless be inclined as has been seen above.

It can also be seen from this figure that one of the short sides 4 of the annular outer female component 2 is provided with a slot 18 and that the same applies 17 to one of the short sides of the annular inner male component 1. The two slots are situated on the same side of the cage and are arranged coincident with each other, by which means it is possible to insert a tool in order to spread the cases apart from each other (movement H in FIG. 3). The tool passes through the slot 18 of the annular outer female component 2 and is able to deploy on the inside, which pushes the annular inner male component 1 and thus separates the components in order to increase the height of the assembly (movement H in FIG. 3). This makes it possible to impart height to the assembly, but also to create the desired lordosis (angle α in FIG. 4) in the case where the components are moved apart more on one side than on the other.

When the desired lordosis and desired height have been obtained by the surgeon, the cages according to the invention are capable not only of maintaining their structure but also of withstanding compression loads and also shearing (forces P and forces F in FIG. 3).

The openings of the upper and lower faces of the cages according to the invention allow bone graft to be inserted therein in order to bring about fusion.

FIG. 6 shows a longitudinal sectional view along the length of a cage according to the invention, of which the inner and outer surfaces of the annular inner male component 1 and of the annular outer female component 2 have a configuration different than that of the preceding cage. The slots of both components have not been shown.

To withstand the shearing stresses (F in FIG. 3), the annular inner male component 1 seen in profile has its two ends describing a circle of radius (R) such that the contacts between the two components are always effective when the cage is opened and there is lordosis. It may be stated that the arcs arranged on each side of the cage describe substantially the same theoretical circle.

To perform this function, the short sides of the annular inner male component 1 can have a cylindrical shape, the axis of the cylinder being horizontal, and can come into contact with plane faces constituting the inner surface of the short sides of the annular outer female component 2, as is shown here in FIG. 6.

Preferably, the short sides of the annular inner male component 1 are dome-shaped, for example hemispherical, and are in contact with surfaces in the shape of semi-circular gutters constituting the inner surface of the short sides of the annular outer female component 2, as is shown in FIGS. 1 to 5.

As is shown in FIG. 7, a cage has also been produced which is of the same general structure but in which the flexible tabs 21, supporting a single blade 20, are arranged on the annular inner male component 1, whereas the surface of the annular outer female component 2 facing it is smooth, and sliding is thereby obtained in the direction of introduction and wedging is obtained in the opposite direction. The flexible tabs 21 are not arranged parallel to the wall of the long sides of the annular inner male component 1, but substantially perpendicular thereto. Moreover, they slope gently downward when looking from the inside to the outside of the component. The introduction of the inner male component into the outer female component is thus made easier, but the displacement in the opposite direction and thwarted. As is shown in the figure, a greater clearance of the component is possible in the direction of introduction than in the direction of withdrawal, where the flexible tab 21 comes quickly into abutment against the rest of the annular inner male component 1. This type of tab and more particularly designed to cooperate by wedging on an annular outer female component 2. When blocking is obtained by wedging, the annular outer female component 2 does not need to have series 19 of blades arranged on the inner faces and it can therefore be smooth on the inside.

FIG. 8 shows, at the top, that the inner faces of the long sides of the annular outer female component 2 and the outer faces of the long sides of the annular inner male component 1 are each provided with devices that each constitute a system of irreversible elastic engagement in multiple positions. To this end, the annular outer female component 2 is provided, on the inner faces of its long sides, with two series 19 of blades that are inclined in parallel like louvers (which would not be openworked in the model shown but can be openworked) along practically the entire length of the long sides.

These inclined blades are also arranged parallel to the upper surface 12 and to the lower surface 14 of the annular outer female component 2. In vertical section perpendicular to the surface of the long side on which they are arranged, these series of blades have a sawtooth structure as shown in FIG. 9. In a variant not shown, several opposite pairs of series 19 of blades are arranged on the inner faces of the long sides of the annular outer female component 2.

To cooperate with these series 19 of inclined blades, the outer faces of the inner male component 1 also present series 20 of blades which are inclined but are of an inverse configuration and independent of one another. These series of blades are arranged on flexible tabs 21.

The system is made irreversible by using a surface of the blades that is perpendicular to the direction of movement in the sense of disassembly, while maintaining a contact surface inclined in the direction of introduction of the inner female component 1 into the outer female component 2.

As a result of this structure, it is possible to irreversibly vary the total height of the cage and the front/rear and left/right angles between the outer surfaces 11/14.

It will also be noted that the slot 17 formed in a short side of the annular inner male component 1 is elongate and open at the lower part of this component.

FIG. 9, which is a transverse sectional view of FIG. 8, except that the annular inner male component 1 has been introduced into the annular outer female component 2 from below, shows clearly the cooperation between the independent blades 20 of the annular inner male component 1 and the series 19 of blades arranged on the inner faces of the annular outer female component 2.

FIG. 10, similar to that of FIG. 4, illustrates another embodiment of the blocking of the longitudinal displacement of the annular inner male component (1) with respect to the annular outer female component (2). In the embodiment shown here, in which the height adjustment device is not depicted, a pin 22 has been arranged in a median position with respect to the length of the cage, in the long sides 7, 9 of the inner male component 1. These pins can move in vertical translation in an oblong hole 23 passing through each of the long sides 8, 10 of the outer female component 2. In another embodiment, a single pin has been used that extends from one long side of the cage to the other long side of the cage.

FIG. 11 illustrates another embodiment of a device for adjusting the height of the cage by acting individually on one end and the other end of the longitudinal axis of the annular inner male component (1). The device used here comprises two elongate rotary shafts, namely inner shaft 30 and outer shaft 31, with concentric and independent axes, each shaft 30, 31 having a cam with flattened segments 32, 33 and being able to be turned independently of the other one. The inner elongate shaft 30 is received at one of its ends in a blind bore 34 provided in the short side of the annular outer female component 2 opposite the slots 17, 18. The cams 32, 33 bear against an inner upper surface 35 of the annular inner male component 1.

In the perspective view of the shafts in FIG. 12, it will be noted that the flattened segments 36 of each cam 32, 33 are progressively spaced farther away from the axis A-B. Blocking is effected each time a flattened segment 36 comes to bear against an inner upper surface 35 of the annular inner male component.

At its end accessible through the slot 18 of the female component, the inner shaft 30 is provided with a recess for a Torx® bit, as shown, an Allen® bit or similar, with a view to permitting its rotation. With this same aim, the outer shaft 31 for its part is provided with cuttings or channels on the same side permitting the use, for example, of a flat-head or cross-head screwdriver.

By turning both shafts, it is possible to increase the height of the cage in an equal manner along the longitudinal axis. By turning only one of the two shafts, the height is increased on only the side of the longitudinal axis where the cam acts, and it is thus possible to obtain lordosis. By turning one more than the other, it is possible to simultaneously increase the height of the cage and produce lordosis.

1. An intervertebral cage comprising two components, namely
an annular inner male component (1), which has two short sides (3, 5) and two long sides (7, 9), hence four outer faces and four inner faces, and an upper surface (11) and a lower surface (13), and
an outer female component (2), which is likewise annular and has two short sides (4, 6) and two long sides (8, 10), hence four outer faces and four inner faces, and an upper surface (12) and a lower surface (14),
the upper surface (11) of the annular inner male component and the lower surface (14) of the annular outer female component forming outer surfaces that determine the height of the cage, said annular inner male component (1) engaging in the annular outer female component (2), each of these two components having the general shape of a parallelepiped, hence a longitudinal axis, the annular inner male component (1) being able to slide with respect to the annular outer female component (2) and having a device for blocking a longitudinal displacement of the annular inner male component (1) with respect to the annular outer female component (2),
the intervertebral cage being provided with a device which adjusts the height of the cage and acts individually on one end and the other end of the longitudinal axis of the annular inner male component (1),
a short side of the annular inner male component and a corresponding short side of the annular outer female component each being provided with a slot (17, 18) for the insertion of an adjustment tool which passes through the annular outer female component in order to actuate the annular inner male component,
as a consequence of which it is possible to increase the height of the cage and modify the angle between the outer surfaces (11, 14).

2. The cage as claimed in claim 1, wherein the device for adjusting the height of the cage comprises two elongate rotary shafts (30, 31) with concentric and independent axes, each shaft having a cam (32, 33) with flattened segments and being able to be turned independently of the other.

3. The cage as claimed in claim 1, wherein the outer faces of the long sides of the annular inner male component are provided with a pair of flexible tabs (21), each of these being provided with a blade (20) protruding from the outer general surface of said long sides, and cooperate by wedging with the inner surface of the long sides of the annular outer female component.

4. The cage as claimed in claim 1, wherein the outer faces of the long sides of the annular inner male component or the inner faces of the long sides of the annular outer female component, or all of these faces, are each provided with two or more devices (19, 20, 21) that each constitute a system of elastic engagement.

5. The cage as claimed in claim 1, wherein the outer face of the short sides (3, 5) of the inner male component is convex and bears on the inner face of the short sides (4, 6) of the annular outer female component.

6. The cage as claimed in claim 1, wherein the short sides (3, 5) of the annular inner male component have the shape of a semi-cylindrical surface, while the inner surfaces of the short sides (4, 6) of the annular outer female component (2) are plane faces.

7. The cage as claimed in claim 1, wherein the short sides (3, 5) of the annular inner male component have the shape of a dome, and the inner surface of the short sides (4, 6) of the outer female component has the shape of semi-circular gutters that are arranged vertically.

8. The cage as claimed in claim 1, wherein pins (22) are arranged in the long sides of the inner male component, which pins (22) cooperate with an oblong hole (23) passing through each of the long sides (8, 10) of the outer female component.

9. The cage as claimed in claim 2, wherein the outer face of the short sides (3, 5) of the inner male component is convex and bears on the inner face of the short sides (4, 6) of the annular outer female component.

10. The cage as claimed in claim 3, wherein the outer face of the short sides (3, 5) of the inner male component is convex and bears on the inner face of the short sides (4, 6) of the annular outer female component.

11. The cage as claimed in claim 4, wherein the outer face of the short sides (3, 5) of the inner male component is convex and bears on the inner face of the short sides (4, 6) of the annular outer female component.

12. The cage as claimed in claim 2, wherein the short sides (3, 5) of the annular inner male component have the shape of a semi-cylindrical surface, while the inner surfaces of the short sides (4, 6) of the annular outer female component (2) are plane faces.

13. The cage as claimed in claim 3, wherein the short sides (3, 5) of the annular inner male component have the shape of a semi-cylindrical surface, while the inner surfaces of the short sides (4, 6) of the annular outer female component (2) are plane faces.

14. The cage as claimed in claim 4, wherein the short sides (3, 5) of the annular inner male component have the shape of a semi-cylindrical surface, while the inner surfaces of the short sides (4, 6) of the annular outer female component (2) are plane faces.

15. The cage as claimed in claim 5, wherein the short sides (3, 5) of the annular inner male component have the shape of a semi-cylindrical surface, while the inner surfaces of the short sides (4, 6) of the annular outer female component (2) are plane faces.

16. The cage as claimed in claim 2, wherein the short sides (3, 5) of the annular inner male component have the shape of a dome, and the inner surface of the short sides (4, 6) of the outer female component has the shape of semi-circular gutters that are arranged vertically.

17. The cage as claimed in claim 3, wherein the short sides (3, 5) of the annular inner male component have the shape of a dome, and the inner surface of the short sides (4, 6) of the outer female component has the shape of semi-circular gutters that are arranged vertically.

18. The cage as claimed in claim 4, wherein the short sides (3, 5) of the annular inner male component have the shape of a dome, and the inner surface of the short sides (4, 6) of the outer female component has the shape of semi-circular gutters that are arranged vertically.

19. The cage as claimed in claim 5, wherein the short sides (3, 5) of the annular inner male component have the shape of a dome, and the inner surface of the short sides (4, 6) of the outer female component has the shape of semi-circular gutters that are arranged vertically.

20. The cage as claimed in claim 2, wherein pins (22) are arranged in the long sides of the inner male component, which pins (22) cooperate with an oblong hole (23) passing through each of the long sides (8, 10) of the outer female component.
